SEQUENCE LISTING (14 sequences)

SEQ ID NO: 1 - DNA, 3476 bp, synthetic construct
  source 1..3476: mol_type = other DNA
atgatgggttatgagtatattttgggaggcattatagcttctagcttggtgcttgtgtttgttatatatggttctgtatcaaagaggaaggccaaaagttcagtacatgcagaaagtaatggtggtagtattataaggacatcaccagaaaatggaaaccaccatcaagaaatctcagaaactacggacgtcatcattgtgggtgctggggttgctggtgcagctcttgcttacactcttggaaaggatggacggcgagtgcatgtaattgaaagggatttgagtgaacctgacaggatcgtgggtgaattgctacaacctgggggctatctgaggttaattgagttgggccttcaggattgtgtggatgaaattgattcacagcaagtctttggctatgcacttttcaaggatgggaaacatactcgtctctcttaccccttggagaagtttcactcagatgtctctggcagaagctttcataatgggcgctttattcagaggatgcgggagaaagccgcctccattcccaatgtacgattggagcaaggaacagtcacttccctaattgaagagaaggggacaattaaaggtgtgcactacaaaacgaaggatagtcaagaattatcagcttgtgcaccccttaccgttgtttgtgatggctgtttctcaaacttgcgccgatctctttgtaatcctaaggtagatgttccctctcatttcgttggcttaattttggagagttgtgaacttccttatgctaatcatggccatgtcatactgggagatccttcgccagtcttattttatcgcatcagcagcactgaagttcggtgcttagttgatatacctggtcaaaaagtttctcctgttggtaaaggtggaatggccaactaccttagggctatggtggcttcccagattccacccgagcttcgtgatgctttcatatctgcaattgatcgaggaaatatcagaaccatgcctaacagtagcatgcctgcctcaccttatcccacaccaggtgcccttctaatgggagatgccttcaatatgcgtcaccctttaaccggaggaggaatgactgtggctttatctgacattgttgtgctaagggacttacttaaaccccttcatgatctccatgatgcttctgctctttgcagataccttgaatcattctacaccctacgcaagcctgtagcatctacaataaacacactagccggggcattgtacaaagtttttttatttaagccttggaggtgttttctcagatggaccaatagctctactctctggtctaaatcctcgtccattaagcttggttctccacttctttgccgtggctatatatggcgttggtcgcttactcataccattcccttctccaaaacgcatgtggattggagctagattgatttccggtgcatctgctatcattttccccattatcaaggctgaaggaattagacaaatgttcttcccagtaactgtgccagcatattacagaacaccccctaccaatttggatgattaaaggtctgtcttattttgaatctcatcagccagataaatatttaatactagctctgctggaatgattttgggcgcatgcttctgtgttattgtatcttattaggagcttctttgtaaattctgtaacatactaattactaattagtaagcagattagtcaaaactatgttgttggtttctggatatgaataacctgtcagggtttccttgatattaaatatgagtgttgccccatttggctctttattgttctattaattaagtcacaatatatttagaagtcatttttatgataatgtgtgtgcctccattttccttggaccttatttatgtcaatttcaccctgaagaattttttttgttagtttttagtaattgtggtttactagtgcggtatattaatcatccaaattggtagggggtgttctgtaatatgctggcacagttactgggaagaacatttgtctaattccttcagccttgataatggggaaaatgatagcagatgcaccggaaatcaatctagctccaatccacatgcgttttggagaagggaatggtatgagtaagcgaccaacgccatatatagccacggcaaagaagtggagaaccaagcttaatggacgaggatttagaccagagagtagagctattggtccatctgagaaaacacctccaaggcttaaataaaaaaactttgtacaatgccccggctagtgtgtttattgtagatgctacaggcttgcgtagggtgtagaatgattcaaggtatctgcaaagagcagaagcatcatggagatcatgaaggggtttaagtaagtcccttagcacaacaatgtcagataaagccacagtcattcctcctccggttaaagggtgacgcatattgaaggcatctcccattagaagggcacctggtgtgggataaggtgaggcaggcatgctactgttaggcatggttctgatatttcctcgatcaattgcagatatgaaagcatcacgaagctcgggtggaatctgggaagccaccatagccctaaggtagttggccattccacctttaccaacaggagaaactttttgaccaggtatatcaactaagcaccgaacttcagtgctgctgatgcgataaaataagactggcgaaggatctcccagtatgacatggccatgattagcataaggaagttcacaactctccaaaattaagccaacgaaatgagagggaacatctaccttaggattacaaagagatcggcgcaagtttgagaaacagccatcacaaacaacggtaaggggtgcacaagctgataattcttgactatccttcgttttgtagtgcacacctttaattgtccccttctcttcaattagggaagtgactgttccttgctccaatcgtacattgggaatggaggcggctttctcccgcatcctctgaataaagcgcccattatgaaagcttctgccagagacatctgagtgaaacttctccaaggggtaagagagacgagtatgtttcccatccttgaaaagtgcatagccaaagacttgctgtgaatcaatttcatccacacaatcctgaaggcccaactcaattaacctcagatagcccccaggttgtagcaattcacccacgatcctgtcaggttcactcaaatccctttcaattacatgcactcgccgtccatcctttccaagagtgtaagcaagagctgcaccagcaaccccagcacccacaatgatgacgtccgtagtttctgagatttcttgatggtggtttccattttctggtgatgtccttataatactaccaccattactttctgcatgtactgaacttttggccttcctctttgatacagaaccatatataacaaacacaagcaccaagctagaagctataatgcctcccaaaatatactcataacccatcat

SEQ ID NO: 2 - protein, 821 aa, Aspergillus nidulans
MADTRRRQNHSCDPCRKGKRRCDAPENRNEANENGWVSCSNCKRWNKDCTFNWLSSQRSKAKGAAPRARTKKARTATTTSEPSTSAATIPTPESDNHDAPPVINSHDALPSWTQGLLSHPGDLFDFSHSAIPANAEDAANVQSDAPFPWDLAIPGDFSMGQQLEKPLSPLSFQAVLLPPHSPNTDDLIRELEEQTTDPDSVTDTNSVQQVAQDGSLWSDRQSPLLPENSLCMASDSTARRYARSTMTKNLMRIYHDSMENALSCWLTEHNCPYSDQISYLPPKQRAEWGPNWSNRMCIRVCRLDRVSTSLRGRALSAEEDKAAARALHLAIVAFASQWTQHAQRGAGLNVPADIAADERSIRRNAWNEARHALQHTTGIPSFRVIFANIIFSLTQSVLDDDEQHGMGARLDKLLENDGAPVFLETANRQLYTFRHKFARMQRRGKAFNRLPGGSVASTFAGIFETPTPSSESPQLDPVVASEEHRSTLSLMFWLGIMFDTLSAAMYQRPLVVSDEDSQISSASPPRRGAETPINLDCWEPPRQVPSNQEKSDVWGDLFLRTSDSLPDHESHTQISQPAARWPCTYEQAAAALSSATPVKVLLYRRVTQLQTLLYRGASPARLEAAIQRTLYVYNHWTAKYQPFMQDCVANHELLPSRIQSWYVILDGHWHLAAMLLADVLESIDRDSYSDINHIDLVTKLRLDNALAVSALARSSLRGQELDPGKASPMYRHFHDSLTEVAFLVEPWTVVLIHSFAKAAYILLDCLDLDGQGNALAGYLQLRQNCNYCIRALQFLGRKSDMAALVAKDLERGLNGKVDSFL

SEQ ID NO: 3 - DNA, 246 bp, Aspergillus nidulans
  source 1..246: mol_type = genomic DNA
cgggatagttccgacctaggattggatgcatgcggaaccgcacgagggcggggcggaaattgacacaccactcctctccacgcaccgttcaagaggtacgcgtatagagccgtatagagcagagacggagcactttctggtactgtccgcacgggatgtccgcacggagagccacaaacgagcggggccccgtacgtgctctcctaccccaggatcgcatccccgcatagctgaacatctatataa

SEQ ID NO: 4 - DNA, 33 bp, Cestrum Yellow Curling Leaf Virus
  source 1..33: mol_type = genomic DNA
tacttagcccctccctcattgttaagggagcaa

SEQ ID NO: 5 - protein, 525 aa, Glycine max
MVDPYVLGWIICAVLSLVALRNFAFARKNRCHSSETDATRRAENVTTAAGECRSSSRDGDVDVIIVGAGVAGSALAHTLGKDGRRVLVIERDLSEQDRIVGELLQPGGYLKLIELGLEDCVEKIDAQLVFGYALFKDGKHTRLSYPLEKFHSDVAGRSFHNGRFIQRMREKAASLSNVRLEQGTVTSLLEEKGVIKGVHYKTKDSQELSACAPLTVVCDGCFSNLRRSLCNPKVDVPSHFVGLILESCELPYANHGHVILGDPSPVLFYRISSSEIRCLVDVPGQKVPSISNGEMTNYLKTVVAPQIPPELHDSFVAAVDKGNIRTMPNRSMPAAPYPTPGALLMGDAFNMRHPLTGGGMTVALSDIVVLRNLLRPLRDLNDAPGLCKYLESFYTLRKPVASTINTLAGALYKVFCASPDPARKEMRQACFDYLSLGGLFSEGPVSLLSGLNPRPLSLVLHFFAVAIYGVGRLLLPFPSPKRMWIGVRLISSASGIILPIIKAEGVRQMFFPATVPAYYRNPPAQ

SEQ ID NO: 6 - protein, 534 aa, Glycine max
MDYQYILGGIMACSFAFLYVVYSFGAKVTDSSSIHVKSNECAKISSENGICSHEDAGSADIIIVGAGVAGSALACTLGKDGRRVHVIERDLSEPDRIVGELLQPGGYLKLIELGLEDCVDEIDAQQVFGYALYKDGKNTKLSYPLENFGSDISGRSFHNGRFIQRMREKASSLPNVKLEQGTVASLLEEKGTITGVHYKTKSGEEFTAKAPLTIVCDGCFSNLRRSLCNPKVEVPSHFVGLVLENCNLPYANHGHVILGDPSPILFYPISSTEIRCLVDVPGQKLPSLGSGEMARYLKTVVAPQVPPELYDSFIAAVDKGNIRTMPNRSMPASPYPTPGALLMGDAFNMRHPLTGGGMTVALSDIVVLRDLLKPLHDLHDASALCRYLESFYTLRKPVASTINTLAGALYKVFCASPDPARKEMRQACFDYLSLGGVFSDGPIALLSGLNPRPLSLVLHFFAVAIYGVGRLLIPFPSPKRMWIGARLISGASGIIFPIIKAEGVRQMFFPATVPAYYRTPPVHPLNTHEGNTWN

SEQ ID NO: 7 - protein, 513 aa, Glycine max
MDYPYILGGIMACSFAFLYVVYSFGAKKVTDSSSIHVKSNECAKTSSEGGICSSDEDAGSADIIIVGAGVAGAALAYTLGKDGRRVHVIERDLSEPDRIVGELLQPGGYLRLIELGLQDCVDEIDSQQVFGYALYMDGKNTKLSYPLEKFSSDISGRSFHNGRFIQRMREKASSLPNVKLEQGTVTSLLEEKGTITGVHYKIKSGQEFTAKAPLTIVCDGCFSNLRRSLCNPKVDVPSHFVGLVLENCNLPYANHGHVILGDPSPVLFYPISSTEIRCLVDVPGQKLPSLGGGEMACYLKTVVAPQVPPELYDSFIAAIDKGNIRTMPNRSMPASPYPTPGALLMGDAFNMRHPLTGGGMTVALSDIVVLRDLLKPLHDLHDASALCRYLESFYTLRKPVASTINTLAGALYKVFCASPDPARKEMRQACFDYLSLGGVFSDGPIALLSGLNPRPLSLVLHFFAVAIYGVGRLLIPFPSPKRMWIGARLISVSTSHFPFCSNNIQNISHITYC

SEQ ID NO: 8 - protein, 525 aa, Glycine max
MDYPYILGGIMACSFAFLYVVYSFGAKKVTDSSSIHVKSNECAKTSSEGGICSSDEDAGSADIIIVGAGVAGAALAYTLGKDGRRVHVIERDLSEPDRIVGELLQPGGYLRLIELGLQDCVDEIDSQQVFGYALYMDGKNTKLSYPLEKFSSDISGRSFHNGRFIQRMREKASSLPNVKLEQGTVTSLLEEKGTITGVHYKIKSGQEFTAKAPLTIVCDGCFSNLRRSLCNPKVDVPSHFVGLVLENCNLPYANHGHVILGDPSPVLFYPISSTEIRCLVDVPGQKLPSLGGGEMACYLKTVVAPQVPPELYDSFIAAIDKGNIRTMPNRSMPASPYPTPGALLMGDAFNMRHPLTGGGMTVALSDIVVLRDLLKPLHDLHDASALCRYLESFYTLRKPVASTINTLAGALYKVFCASPDPARKEMRQACFDYLSLGGVFSDGPIALLSGLNPRPLSLVLHFFAVAIYGVGRLLIPFPSPKRMWIGARLISGASGIIFPIIKAEGVRQMFFPATVPAYYRTPPVH

SEQ ID NO: 9 - protein, 529 aa, Glycine max
MGYEYLLGGIIASSLVLVFVVYGSVSKKMTKGSVVHAESNGGSIIRTSSENRNHHQEISETTDIIIVGAGVAGAALAYTLGKEGRRVHVIERDLTEPDRIVGELLQPGGYLKLIELGLQDCVDEIDAQQVFGYALYKDGKNTKLSYPLEKFASDVSGRSFHNGRFIQRMREKASSLPNVKLEQGTVTSLLEENGIIKGVNYKSKSGQEVTAKASLTIVCDGCFSNLRRSLCNPKVDVPSHFVGLVLENCNLPYANHGHVILSDPSPILFYPISSSEIRCLVDVPGQKLPSLGNGDMARYLKTVVAPQVPPELRESFIAAVEKGNMRSMPNRSMPASPYPTPGALLMGDAFNMRHPLTGGGMTVALSDIVLLRNLLRPLHDLHDANALCKYLESFYTLRKPVASTINTLAGALYKVFCASPDPASKEMRQACFDYLSLGGVFSDGPIALLSGLNPRPLSLVLHFFAVAIYGVGRLLIPFPSPKRMWIGARLISGASAIIFPIIKAEGIRQMFFPVTVPAYYRTPPTNLDD

SEQ ID NO: 10 - protein, 526 aa, Glycine max
MEDPYVLGWILCSVLSLLALYSLAFGGAGRRRASPEKRNELSETVTTSAGECRSEKRNGDADVIIVGAGVAGAALAHTLGKDGRRVHVIERDLSEPDRIVGELLQPGGYLKLIELGLEDCVDKIDAQQVFGYALFKDGKHTRLSYPLEKFHSDVSGRSFHNGRFIQRMREKAASIPNVRLEQGTVTSLIEEKGTIKGVQYKTKDGQELATYAPLTIVCDGCFSNLRRSLCNPKVDIPSCFVGLVLENCELPCANHGHVILGDPSPILFYPISSTEIRCLVDVPGQKVPSISNGEMGKYLKTTVAPQIPPQLYDAFIAAVDKGNIRTMPNRSMPADPHPTPGALLMGDAFNMRHPLTGGGMTVALSDIVVLRNLLRPLSDLNDAPTLCKYLESFYTLRKPVASTINTLAGALYKVFCASPDQARKEMRQACFDYLSLGGLFSEGPVSLLSGLNPRPLSLVLHFFAVAIYGVGRLLLPFPSPKRVWIGARLISGASAIILPIIKAEGIRQMFFPATVPAYYRTPPVAQ

SEQ ID NO: 11 - protein, 529 aa, Glycine max
MMGYEYILGGIIASSLVLVFVIYGSVSKRKAKSSVHAESNGGSIIRTSPENGNHHQEISETTDVIIVGAGVAGAALAYTLGKEGRRVHVIERDLTEPDRIVGELLQPGGYLKLIELGLQDCVGEIDAQPVFGYALYKDGKNTKLSYPLENFASDVSGRSFHNGRFIQRMREKASSLPNVKLEQGTVTFLLEEDRIIKGVNFKTKSGQELTAKAPLTIVCDGCFSNLRRSLCNPKVDVPSHFVGLVLENCNLPYANHGHVILGDPSPILFYPISSTEIRCLVDVPGHKLPSLGNGDMARYLKTVVAPQVPPELRDSFIAAVEKGNIRSMPNRSMPASPYPTPGALLMGDAFNMRHPLTGGGMTVALSDIVLLRNLLRPLHDLHDANALCKYLESFYTLRKPVASTINTLAGALYKVFCASPDPASKEMRQACFDYLSLGGVFSDGPIALLSGLNPRPLSLVLHFFAVAIYGVGRLLIPFPSPKRMWIGARLISGASAIIFPIIKAEGIRQMFFPVTVPAYYRTPPTNLED

SEQ ID NO: 12 - protein, 548 aa, Glycine max
MLPVSSLSLSHHKTTTTIAEATCHCAFFKFSFNKPHHHRTPLLFPNHLPNASPPLLRTTTSLRRNAAADPPPGFSGVADVIVVGAGVAGSALAYTLAKDGRCVHVIERDLREPDRIVGELLQPGGYLKLIELGLQDCLEQIDAQRVLGYVLFKDGKKTKLPYPLENFHADVAGRSFHNGRFIQRMREKAATLHHVRMEQGTVTSLLEQDNGTIIGVQYKTKDGQKLKAYAPFTIVCDGCFSNLRRSLCYPKVEVPSCFVGLVLENCQLPLENYGHVILADPSPILFYRISSTEVRCLVDIPGQKVSPVGKGGMANYLRAMVASQIPPELRDAFISAIDRGNIRTMPNSSMAADPYPTPGALLMGDAFNMRHPLTGGGMTVALSDIVVLRNLLKPICDLNDATSLCRHLESFYILRKPMASTINTLAGALYKVFSTSPNEARKELRQACFDYLSLGHFFSSGPIALLSGLNPSPLNLILHFFAVALYGVGRLLLPFPSFERTWIGAKIVLCAFGIIFPIIKGEGVRQMFFPTSIPAFYRAPPDRWCIEE

SEQ ID NO: 13 - protein, 453 aa, Glycine max
MLCDGRCVHVIERDLREPDRIVGELLQPGGYLKLIELGLQDCLEQIDAQRVLGYVLFKDGKKTKLPYPLENFHADVAGRSFHNGRFIQRMREKAATLHHVRMEQGTVTSLLEQDNGTIIGVQYKTKDGQKLKAYAPFTIVCDGCFSNLRRSLCYPKVEVPSCFVGLVLENCQLPLENYGHVILADPSPILFYRISSTEVRCLVDIPGQKVSPVGKGGMANYLRAMVASQIPPELRDAFISAIDRGNIRTMPNSSMAADPYPTPGALLMGDAFNMRHPLTGGGMTVALSDIVVLRNLLKPICDLNDATSLCRHLESFYILRKPMASTINTLAGALYKVFSTSPNEARKELRQACFDYLSLGHFFSSGPIALLSGLNPSPLNLILHFFAVALYGVGRLLLPFPSFERTWIGAKIVLCAFGIIFPIIKGEGVRQMFFPTSIPAFYRAPPDRWCIEE

SEQ ID NO: 14 - protein, 364 aa, Glycine max
MREKAATLHHVRMEQGTVTSLLEQDNGTIIGVQYKTKDGQKLKAYAPFTIVCDGCFSNLRRSLCYPKVEVPSCFVGLVLENCQLPLENYGHVILADPSPILFYRISSTEVRCLVDIPGQKVSPVGKGGMANYLRAMVASQIPPELRDAFISAIDRGNIRTMPNSSMAADPYPTPGALLMGDAFNMRHPLTGGGMTVALSDIVVLRNLLKPICDLNDATSLCRHLESFYILRKPMASTINTLAGALYKVFSTSPNEARKELRQACFDYLSLGHFFSSGPIALLSGLNPSPLNLILHFFAVALYGVGRLLLPFPSFERTWIGAKIVLCAFGIIFPIIKGEGVRQMFFPTSIPAFYRAPPDRWCIEE

(19) United States
(12) Patent Application Publication
(10) Pub. No.: US 2025/0059546 A1
(43) Pub. Date: Feb. 20, 2025

(54) ENHANCED PRODUCTION OF SQUALENE IN PLANT SYSTEMS

(71) Applicant: THE REGENTS OF THE UNIVERSITY OF COLORADO A BODY CORPORATE, Denver, CO (US)

(72) Inventors: Brian DEDECKER, Boulder, CO (US); Simon KALMUS, Boulder, CO (US); David BIRKHAEUSER, Boulder, CO (US); Nickles BADGER, Boulder, CO (US)

(21) Appl. No.: 18/925,537

(22) Filed: Oct. 24, 2024

Related U.S. Application Data
(63) Continuation-in-part of application No. PCT/US2023/066309, filed on Apr. 27, 2023.
(60) Provisional application No. 63/335,292, filed on Apr. 27, 2022.

(51) Int. Cl. C12N 15/113; C12N 15/82

(52) U.S. Cl. CPC C12N 15/1137; C12N 15/8205; C12N 15/8243; C12N 2310/122; C12N 2310/14

(57) ABSTRACT

Systems, methods and compositions for the inducible production of squalene in plants seeds, in part by downregulating the endogenous sterol production pathway, for example through heterologous expression of one or more inhibitory RNA molecules, and in a preferred embodiment a novel hairpin multiple RNAi molecules that target paralogs of squalene epoxidase (SQE). Downregulating SQE causes a buildup of squalene by both blocking downstream conversion of squalene into sterols and releasing the negative feedback loop that sterols have with the upstream MVA pathway.

### CROSS-REFERENCE TO RELATED APPLICATIONS

This International PCT application claims the benefit of and priority to U.S. Provisional Application No. 63/335,292 filed Apr. 27, 2022, the specification, claims and drawings of which are incorporated herein by reference in their entirety.

### SEQUENCE LISTING

The instant application contains contents of the electronic sequence listing (90245-00781-Sequence-Listing.xml; Size: 22,640 bytes; and Date of Creation: Apr. 27, 2023) is herein incorporated by reference in its entirety.

### FIELD OF INVENTION

The present invention relates to the production of therapeutically and commercially relevant compounds in transgenic plants. In particular, the present invention relates to novel systems, methods and compositions for the production of triterpene compounds, such as squalene in transgenic plants, namely soybeans.

### BACKGROUND

Squalene is a high value compound used as an adjuvant for vaccines and as an additive in the cosmetics industry. The global squalene market size was valued at $114.5 million in 2020 and is projected to reach $241.6 million by 2030. Squalene is produced naturally in plants, but not at sufficient levels to be commercially viable. Thus, squalene is still harvested unsustainably from sharks. Enhancing the levels of squalene in commercially farmed crops would be desirable both economically and environmentally. Squalene is normally produced in the cytoplasm of plant cells and is shunted into the downstream pathway that makes sterols. The natural production of sterols keeps the levels of squalene in the plant low.

Prior attempts to produce squalene at commercial scale have been met with limited success. For example, researchers sought to increase squalene production in plants involved redirecting the squalene synthetic pathway from the cytosol of a tobacco plant to the plastid compartment where squalene is not metabolized to build sterols. However, such systems were shown to be both commercially and technically limited as they are unable prevent shunting of squalene to endogenous sterol production pathway. As such, there is a long-felt need for a more efficient and commercially scalable plant-based technology platform for squalene production.

### SUMMARY OF THE INVENTION

In one aspect, the present invention relates to the heterologous production of therapeutically and commercially relevant compounds in transgenic plants. In particular, the present invention relates to novel systems, methods and compositions for the production of squalene in transgenic plants, namely soybeans. This method could also be used in seeds harvested from other commonly farmed crops such as corn, rice and rapeseeds. In another preferred aspect, the present invention relates to novel systems, methods and compositions for the production of squalene in transgenic plants, namely soybeans. In another aspect, the present invention relates to the inducible heterologous production of therapeutically and commercially relevant compounds in transgenic plants. In particular, the present invention relates to novel systems, methods and compositions for the inducible production of squalene in transgenic plants, namely soybeans seeds.

In another aspect, the present invention is directed to novel systems, methods, and compositions of upregulating the production of squalene in plants by in part, downregulating the endogenous sterol production pathway. In one preferred embodiment, this is accomplished by transient heterologous expression of a hybrid RNAi construct directed against endogenous paralogs of squalene epoxidase (SQE), also known as squalene monooxygenase, which catalyzes the first enzymatic step in sterol, saponin, and brassinosterioid synthesis. In this embodiment, downregulating SQE causes a buildup of squalene in the plant by both blocking downstream conversion of squalene into sterols and releasing the negative feedback loop that sterols have with the upstream MVA pathway. In one preferred aspect, induction of expression of desired genes during the germination of plant seeds allows this RNAi construct to be expressed only during the processing of harvested seeds and mitigate its toxic effects.

Additional aspects of the inventive technology will be evident from the detailed description and figures presented below.

### BRIEF DESCRIPTION OF THE FIGURES

FIG. 1: Mevalonate pathway engineered to maximize accumulation of squalene by knocking down squalene epoxidase using a multiple paralog hairpin RNAi.

FIG. 2: RNAi construct created by combining 200-300 nucleotide fragments of Soybean SQE paralogs directed to inhibit SQE mRNA. The paralog are identified by reference and their sequences are readily available to those of ordinary skill in the art.

FIG. 3: Mechanism showing inducible nature of squalene production. In the absence of an inducer such as Ethanol in the AlcA/AlcR expression system, development and metabolism proceeds uninhibited. In the presence of the inducer ethanol, the hairpin is expressed causing silencing of squalene epoxidase and subsequent increase in squalene production.

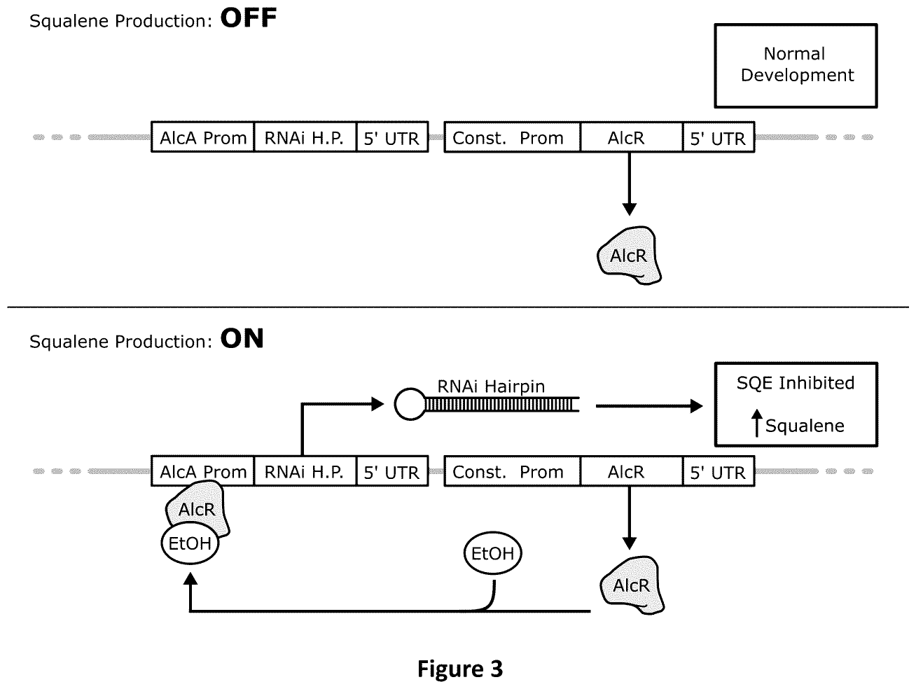

FIG. 4: Induced biosynthesis of squalene in germinating soybeans as shown by HPLC chemical analysis. a. Ethanol induced squalene epoxidase RNAi with Methyl Jasmonate (MeJA): K599 infected germinating soybean root with transgene construct incubated 7 days; 1× MS Salts, 2% Sucrose, 200 mg/mL Cefotaxime 500 mg/L MES, 2% (V/V) Ethanol, 100 uM MeJA. b. Ethanol induced squalene epoxidase RNAi: K599 infected germinating soybean root with transgene construct incubated 7 days; 1× MS Salts, 2% Sucrose, 200 mg/mL Cefotaxime 500 mg/L MES, 2% (V/V) Ethanol. c. Uninduced squalene epoxidase RNAi (without ethanol): K599 infected germinating soybean root with transgene construct incubated 7 days; 1× MS Salts, 2% Sucrose, 200 mg/mL Cefotaxime 500 mg/L MES, 2% (V/V) Ethanol. d-f. Control germinating roots without transfected squalene epoxidase RNAi construct: K599 infected germinating soybean roots, no transgene construct, incubated 7 days; 1× MS Salts, 2% Sucrose, 200 mg/mL Cefotaxime 500 mg/L MES, 2% (V/V) Ethanol, 100uM Methyl Jasmonate.

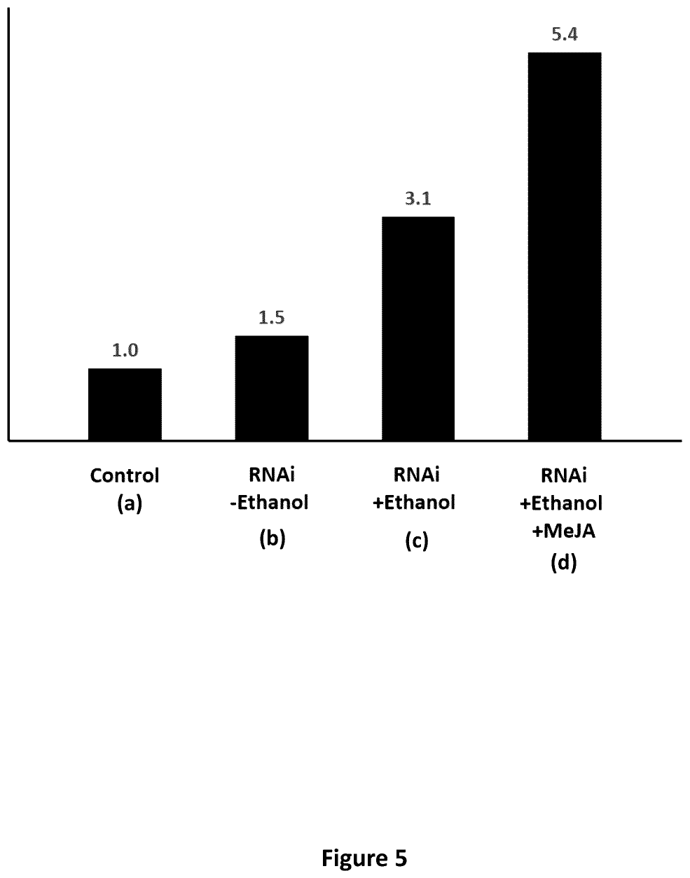

FIG. 5: Induced biosynthesis of squalene in germinating soybeans as shown by HPLC chemical analysis. Quantitation of HPLC signals a. Ethanol induced squalene epoxidase RNAi with MeJA. b. Ethanol induced squalene epoxidase RNAi. c. Uninduced squalene epoxidase RNAi (without ethanol). d. Control germinating root without transfected squalene epoxidase RNAi construct.

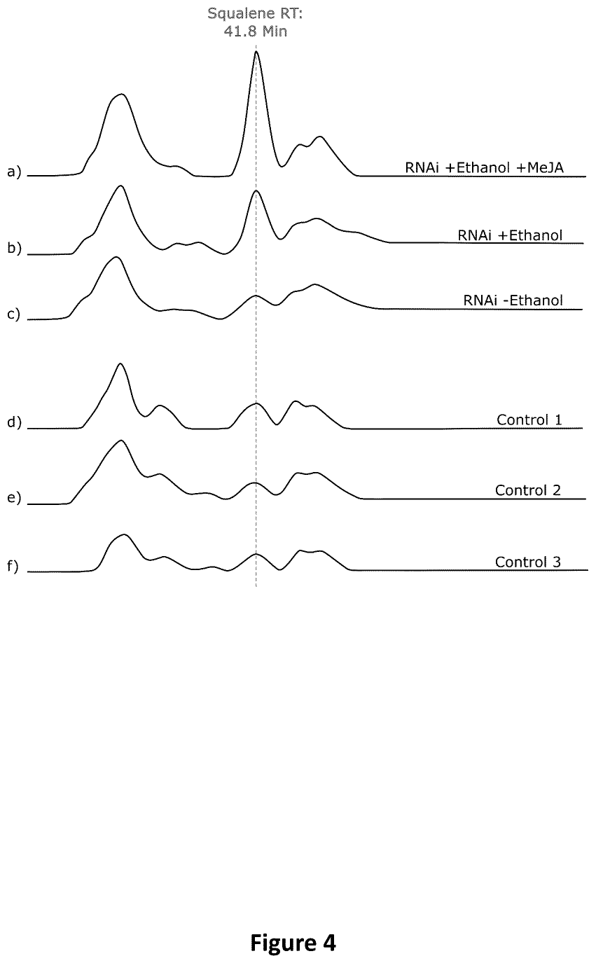

### DETAILED DESCRIPTION OF THE INVENTION

The present invention is directed to novel systems, methods, and compositions of upregulating the production of squalene in plants, and preferably soybeans. In a preferred embodiment, the present invention may upregulate production of squalene in plants by downregulating the endogenous sterol and saponin production pathways. As used herein, “squalene” refers to a compound having the following formula:

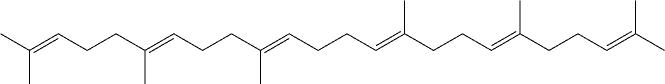 [SMILES: CC(C)=CCC/C(C)=C/CC/C(C)=C/CC/C=C(\C)CC/C=C(\C)CCC=C(C)C]

In a preferred embodiment, downregulating endogenous sterol production may be accomplished through the heterologous expression of one or more inhibitory RNA molecules, and in a preferred embodiment a novel hairpin multiple RNAi molecule according to SEQ ID NO. 1, or a fragment or variant thereof directed to one or more mRNA of the paralogs of SQE.

In a preferred embodiment, the present invention may upregulate production of squalene in plants by downregulating the endogenous sterol production pathway, for example through heterologous expression of one or more inhibitory RNA molecules, and in a preferred embodiment a novel hairpin multiple paralog RNAi molecules according to SEQ ID NO. 1, or a fragment or variant thereof. In this preferred embodiment, the RNAi molecule of the invention targets and inhibits one or more squalene epoxidase paralogs in Glycine Max (soybeans). In this preferred embodiment, the RNAi molecule according to SEQ ID NO. 1, or a fragment or variant thereof, inhibits 7 squalene epoxidase paralogs (and their associated isoforms) in Glycine Max, selected from: Glyma.06g049500 (SEQ ID NO. 5), Glyma.07g185500 (SEQ ID NO. 6), Glyma.08g063700 (SEQ ID NO. 7), Glyma.08g063700-isoform (SEQ ID NO. 8), Glyma.13g253100 (SEQ ID NO. 9), Glyma.14g090400 (SEQ ID NO. 10), Glyma.15g061800 (SEQ ID NO. 11), and Glyma.18g118100 (SEQ ID NO. 12), Glyma.18g118100-isoform 1 (SEQ ID NO. 13), Glyma.18g118100-isoform 2 (SEQ ID NO. 14).

As noted above, the nucleotide sequences encoding the genes necessary to increase production of squalene may be subject to an inducible promoter, such as an AlcA/AlcR inducible expression system. The nucleotide sequences encoding the genes necessary to increase production of squalene may be encoded and expressed in a plant, and preferably a soybean plant, by a heterologous nucleotide sequence comprising one or more expression cassettes, operably linked to promoter(s), encoding one or more of the following heterologous enzymes that upregulate the squalene biosynthesis pathway: a multiple paralog RNAi molecules according to SEQ ID NO. 1, or a fragment or variant thereof targeting the paralogs of SQE.

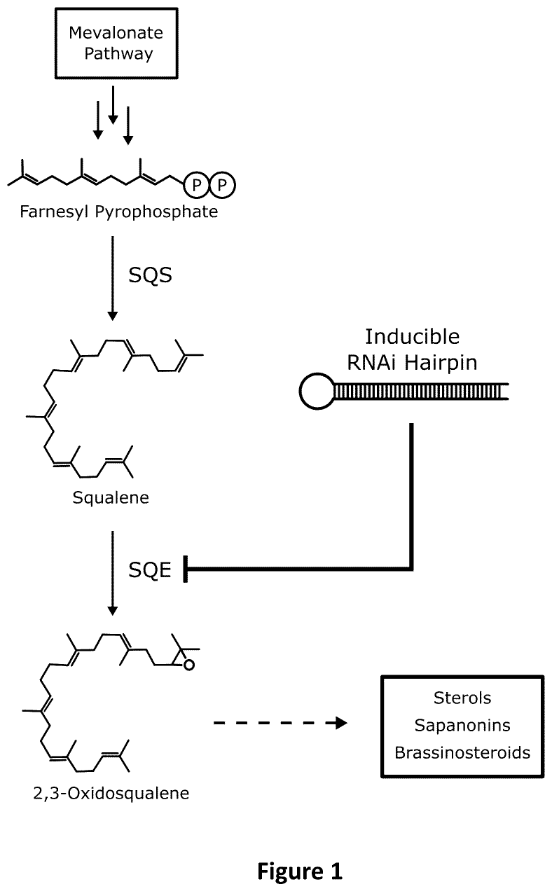

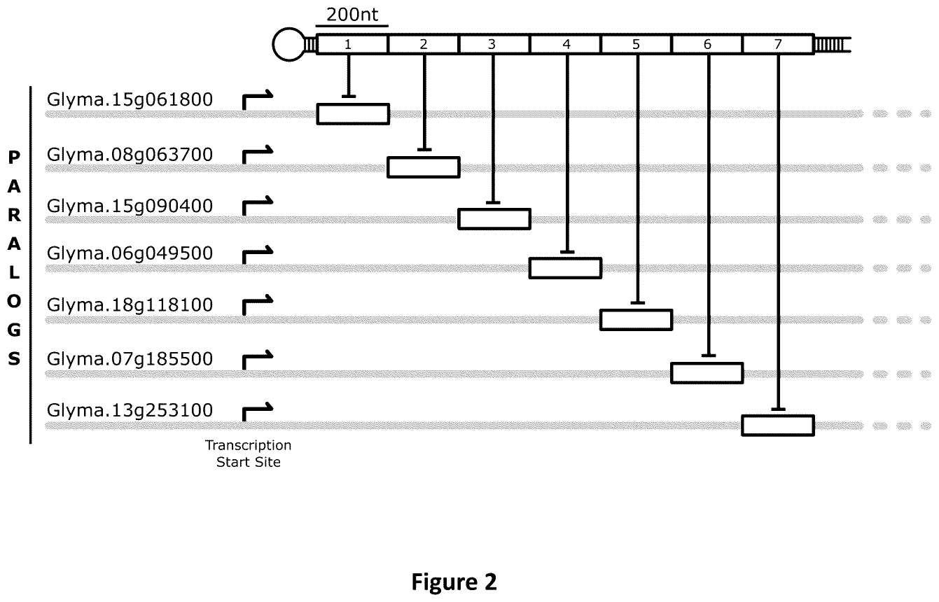

As shown in FIGS. 1 and 2, a long hairpin RNAi was constructed that is targeted to paralogs of squalene epoxidase (SQE) (SEQ ID NO's. 5-14), as well as isoforms thereof, to mitigate downstream metabolism of squalene to sterols. The RNAi sequence was assembled by first identifying SQE paralogs, followed by multiple sequence alignment and subsequent fragment selection which were concatenated in sense and antisense directions with an intron sequence between. Selected target fragments corresponding to different paralogs ranged in length from 200-300 bp. Via the RNAi silencing complex (RISC) pathway, dicer cuts hairpin RNA into 20-24 bp fragments. The RISC complex can utilize the siRNAs from the novel construct as antisense guide RNAs to partially and/or fully inhibit expression of one or more, or preferably all 7 SQE paralogs.

To determine if the inserts had successfully been transfected into the expression vector, DNA isolated from a transfected colony was sequenced via Oxford Nanopore sequencing. In this embodiment, the gene constructs can be inserted into agrobacterium vector for plant, and in particular soybean plant transformation.

Vector construction was performed using golden gate protocol in E. coli for insertion of a novel multiple-paralog RNAi knockdown aimed at transcriptional silencing of squalene epoxidase (SQE) to inhibit downstream metabolism of squalene. An agrobacterium intermediate vector can be utilized for ultimate transformation of SQE-RNAi into soybean seed.

All heterologously expressed coding sequences may be driven by the inducible AlcA/AlcR system with the AlcA promoter sequence according to SEQ ID NO. 3. A 33 base pair spacer (SEQ ID NO. 4) was utilized between the transcription start site and translation start site for all heterologous sequences. Terminators may be used to comprise the 3′ untranslated regions (3′ UTR) of the transgenes.

As noted above, in another embodiment, the invention may include a system for transforming a plant, and preferably a soybean plant to produce a transgenic seed expressing a heterologous nucleotide sequence, operably linked to a promoter, encoding one or more peptides and/or RNAi necessary for the upregulation of squalene production. The genes encoding one or more peptides and/or RNAi necessary for the upregulation of squalene production may be activated by one or more inducible promoters. For example, wild-type (non-engineered) seeds may also be used in this method by transfecting them with the transgenic DNA after germination. In this embodiment, wild-type seeds are harvested and germinated. Once germinated, the seeds are transfected with the transgenic DNA via the standard agrobacterium mediated method. From there, the genes encoding one or more peptides and/or RNAi necessary for the upregulation of squalene production are activated. Known inducible promoter systems have been developed for plants such as the ethanol inducible expression system from Aspergillus Nidulans (AlcR/AlcA). The Aspergillus Nidulans AlcR/AlcA ethanol inducible expression system described in WO2001009357A2, by Syngenta Ltd., (incorporated herein by reference) can be used to induce expression of the synthetic metabolic pathway shortly after seed germination. Other examples of the method may utilize a similar inducible promoter system such as the estradiol dependent XVE system to control the timing of recombinant gene expression.

As noted above, the present invention relates to the expression, processing and delivery of RNA to target cells. As used herein, the term RNA is used as it is in the art and is intended to mean at least one ribonucleic acid molecule. In one embodiment, the RNA is intended to elicit a gene silencing response, or RNA interference (RNAi) in the target cell. For example, the RNA expressed and processed in a plant cell may be double stranded (dsRNA). This dsRNA may be processed in the plant cell into small interfering RNA (siRNA) as described herein and delivered to the target cell.

For example, dsRNA may be heterologously expressed in a yeast cell and processed into siRNA that can be delivered to a target cell. As is well-known, the RNAi silencing complex (RISC) pathway, dicer cuts hairpin RNA, such as SEQ ID NO. 1, into 20-24 bp fragments. As a result, in the invention, the plant cell's RISC complex can utilize the siRNAs from the novel construct as antisense guide RNAs to partially and/or fully inhibit expression of one or more, or preferably the 7 major SQE paralogs (SEQ ID NO's. 5-7, and 9-12).

As is well-known, the use of dsRNA to silence gene expression is not limited to specific messenger RNA (mRNA) sequences for each targeted gene. Rather, one strand of the dsRNA should be perfectly complementary or predominantly complementary to a region of the target mRNA that is targeted for silencing. There are now well-established “rules” and guidelines for design of, for example, siRNA molecules that can target mRNAs for cleavage and therefore gene silencing. For example, one of skill may employ the Ui-Tei rule (Ui-Tei, K., et al., Nucleic Acids Res., 32:936-948 (2004), incorporated by reference), the Reynolds rule (Reynolds, A., et al., Nat. Biotechnol., 22:326-330 (2004), incorporated by reference) or the Amarzguioui rule (Amarzguioui, M and Prydz, H., Biochem. Biophys. Res. Commun., 316:1050-1058 (2004), incorporated by reference) in designing siRNAs for delivery into the target cell.

Accordingly, the heterologous dsRNA expressed in a plant cell may include siRNA having a sequence on one strand of the RNA duplex that follows the Ui-Tei rule, the Reynolds rule or the Amarzguioui rule. For example, an siRNA that has one strand following the Ui-Tei rule includes (a) an A or U at position 1, from 5′ terminus of siRNA guide strand, (b) a G or Cat position 19, (3) having AU in four or more positions of 1-7 of the guide strand, and (d) no long GC stretches of ten or more nucleotides. Additional characteristics of the siRNA sequence may or may not include other aspects of designer siRNA sequences, such as but not limited to having a UU sequence for at least one of the 3′ overhangs, a GC content of between about 30% to about 60%, for example around 50% to 52%. Other characteristics of the siRNA sequence that is delivered to the target cells may or may not include those characteristics noted in Naito, Y. and Ui-Tei, K., Front. Genet., Vol 3, Article 102 (2012).

Generally speaking, the siRNA delivered to the target cell contains one or two strands. Whereas the siRNA is a single strand may be the guide strand i.e., the guide strand, that is perfectly (100%) complementary to a small stretch, about 15-23 bases, to a sequence within the target mRNA. Where the siRNA is a small double stranded RNA, the appropriately sized dsRNA will interact with the host Argonaut-1 (AGO) protein to select the guide strand. Thus, the siRNA delivered to the target cell is not necessarily limited to a specific nucleotide sequence, except that the siRNA that is delivered the cell after delivery may be designed to have one strand that is 100% complementary to between about 15-23 bases of a target mRNA, or may interact with the host AGO peptide to select produce the complementary guide strand.

In additional embodiments, the siRNA that is delivered to the target cells is an siRNA directed to one or more target genes. As used herein, the phrase “directed against” or “directed to” when used in conjunction with RNA means that the RNA comprises at least one strand that is designed to promote gene silencing for a target gene.

A polypeptide or polynucleotide can be expressed in monocot plants and/or dicot plants. Techniques for introducing nucleic acids into plants are known in the art, and include, without limitation, Agrobacterium-mediated transformation, viral vector-mediated transformation, electroporation, and particle gun transformation (also referred to as biolistic transformation). Sec, for example, U.S. Pat. Nos. 5,538,880; 5,204,253; 6,329,571; and 6,013,863; Richards et al., Plant Cell. Rep. 20:48-20 54 (2001); Somleva et al., Crop Sci. 42:2080-2087 (2002); Sinagawa-Garcia et al., Plant Mol Biol (2009) 70:487-498; and Lutz et al., Plant Physiol., 2007, Vol. 145, pp. 1201-1210. In some instances, intergenic transformation of plastids can be used as a method of introducing a polynucleotide into a plant cell. In some instances, the method of introduction of a polynucleotide into a plant comprises chloroplast transformation. In some instances, the leaves and/or stems can be the target tissue of the introduced polynucleotide. If a cell or cultured tissue is used as the recipient tissue for transformation, plants can be regenerated from transformed cultures if desired, by techniques known to those skilled in the art.

Other suitable methods for introducing polynucleotides include electroporation of protoplasts, polyethylene glycol-mediated delivery of naked DNA into plant protoplasts, direct gene transformation through imbibition (e.g., introducing a polynucleotide to a dehydrated plant), transformation into protoplasts (which can comprise transferring a polynucleotide through osmotic or electric shocks), chemical transformation (which can comprise the use of a polybrene-spermidine composition), microinjection, pollen-tube pathway transformation (which can comprise delivery of a polynucleotide to the plant ovule), transformation via liposomes, shoot apex method of transformation (which can comprise introduction of a polynucleotide into the shoot and regeneration of the shoot), sonication-assisted agrobacterium transformation (SAAT) method of transformation, infiltration (which can comprise a floral dip, or injection by syringe into a particular part of the plant (e.g., leaf)), silicon-carbide mediated transformation (SCMT) (which can comprise the addition of silicon carbide fibers to plant tissue and the polynucleotide of interest), electroporation, and electrophoresis. Such expression may be from transient or stable transformations.

The term “homolog” or “variant,” used with respect to an original enzyme or gene of a first family or species, refers to distinct enzymes or genes of a second family or species which are determined by functional, structural or genomic analyses to be an enzyme or gene of the second family or species which corresponds to the original enzyme or gene of the first family or species. Most often, homologs or variant will have functional, structural or genomic similarities. Techniques are known by which homologs of an enzyme or gene can readily be cloned using genetic probes and PCR. Identity of cloned sequences as homolog can be confirmed using functional assays and/or by genomic mapping of the genes. A “fragment” used with respect to an original enzyme or gene refers to a truncated portion of the peptide or gene that still retains its intended function. As used herein, “paralog” refer to genes that are related by replication within a genomc.

The term “operably linked,” when used in reference to a regulatory sequence and a coding sequence, means that the regulatory sequence affects the expression of the linked coding sequence. “Regulatory sequences,” or “control elements,” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell. An “inducible” promoter may be a promoter which may be under environmental control. Tissue-specific, tissue-preferred, cell type specific, and inducible promoters constitute the class of “non-constitutive” promoters. A “constitutive” promoter is a promoter which may be active under most environmental conditions or in most cell or tissue types.

As used herein, the term “transformation” or “genetically modified” refers to the transfer of one or more nucleic acid molecule(s) into a cell. A plant is “transformed” or “genetically modified” by a nucleic acid molecule transduced into the plant when the nucleic acid molecule becomes stably replicated by the plant. As used herein, the term “transformation” or “genetically modified” encompasses all techniques by which a nucleic acid molecule can be introduced into, such as a plant.

The term “vector” refers to some means by which DNA, RNA, a protein, or polypeptide can be introduced into a host. The polynucleotides, protein, and polypeptide which are to be introduced into a host can be therapeutic or prophylactic in nature; can encode or be an antigen; or can be regulatory in nature, etc. There are various types of vectors including virus, plasmid, bacteriophages, cosmids, and bacteria. An “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.” In contrast, as described in the examples herein, a “cassette” is a polynucleotide containing a section of an expression vector of this invention. The use of a cassette assists in the assembly of the expression vectors. An expression vector is a replicon, such as plasmid, phage, virus, chimeric virus, or cosmid, and which contains the desired polynucleotide sequence operably linked to the expression control sequence(s).

As is known in the art, different organisms preferentially utilize different codons for generating polypeptides. Such “codon usage” preferences may be used in the design of nucleic acid molecules encoding the proteins and chimeras of the invention in order to optimize expression in a particular host cell system. For example, all nucleotides of the present invention may be optimized for expression in a select organisms such a Glycine Max.

A polynucleotide sequence is operably linked to an expression control sequence(s) (e.g., a promoter and, optionally, an enhancer) when the expression control sequence controls and regulates the transcription and/or translation of that polynucleotide sequence.

Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Because of the degeneracy of nucleic acid codons, one can use various different polynucleotides to encode identical polypeptides. The Table below, contains information about which nucleic acid codons encode which amino acids.

### Amino Acid Nucleic Acid Codons

| Amino Acid | Nucleic Acid Codons |
|---|---|
| Ala/A | GCT, GCC, GCA, GCG |
| Arg/R | CGT, CGC, CGA, CGG, AGA, AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAC |
| Val/V | GTT, GTC, GTA, GTG |

Moreover, because the proteins are described herein, one can chemically synthesize a polynucleotide which encodes these polypeptides/chimeric proteins. Oligonucleotides and polynucleotides that are not commercially available can be chemically synthesized e.g., according to the solid phase phosphoramidite triester method first described by Beaucage and Caruthers, Tetrahedron Letts. 22:1859-1862 (1981), or using an automated synthesizer, as described in Van Devanter et al., Nucleic Acids Res. 12:6159-6168 (1984). Other methods for synthesizing oligonucleotides and polynucleotides are known in the art. Purification of oligonucleotides is by either native acrylamide gel electrophoresis or by anion-exchange HPLC as described in Pearson & Reanier, J. Chrom. 255:137-149 (1983).

The term “plant” or “plant system” includes whole plants, plant organs, progeny of whole plants or plant organs, embryos, somatic embryos, embryo-like structures, protocorms, protocorm-like bodies (PLBs), and culture and/or suspensions of plant cells. Plant organs comprise, e.g., shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like).

The term “expression,” as used herein, or “expression of a coding sequence” (for example, a gene or a transgene) refer to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

The term “nucleic acid” or “nucleic acid molecules” include single-and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double stranded RNA), siRNA (small interfering RNA), mRNA (messenger RNA), miRNA (micro-RNA), hpRNA (hairpin RNA), tRNA (transfer RNA), whether charged or discharged with a corresponding acetylated amino acid), and cRNA (complementary RNA). The term “deoxyribonucleic acid” (DNA) is inclusive of cDNA, genomic DNA, and DNA-RNA hybrids. The terms “nucleotide sequence” and “nucleotide sequence segment,” or more generally “sequence,” will be understood by those in the art as a functional term that includes both genomic sequences, ribosomal RNA sequences, transfer RNA sequences, messenger RNA sequences, operon sequences, and smaller engineered nucleotide sequences that encoded or may be adapted to encode, peptides, polypeptides, or proteins.

The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide. It should be noted that any reference to a SEQ ID, or sequence specifically encompasses that sequence, as well as all corresponding sequences that correspond to that first sequence. For example, for any amino acid sequence identified, the specific specifically includes all compatible nucleotide (DNA and RNA) sequences that give rise to that amino acid sequence or protein, and vice versa.

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hair-pinned, circular, and padlocked conformations.

The term “sequence identity” or “identity,” as used herein in the context of two nucleic acid or polypeptide sequences, refers to the residues in the two sequences that are the same when aligned for maximum correspondence over a specified comparison window.

The terms “approximately” and “about” refer to a quantity, level, value, or amount that varies by as much as 30%, or in another embodiment by as much as 20%, and in a third embodiment by as much as 10% to a reference quantity, level, value or amount. As used herein, the singular form “a,” “an,” and “the” include plural references unless the context clearly dictates otherwise.

As used herein, “heterologous” or “exogenous” in reference to a nucleic acid is a nucleic acid that originates from a foreign species, or is synthetically designed, or, if from the same species, is substantially modified from its native form in composition and/or genomic locus by deliberate human intervention. A heterologous protein may originate from a foreign species or, if from the same species, is substantially modified from its original form by deliberate human intervention.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a 13C- or 14C-enriched carbon are within the scope of this invention.

Such compounds are useful, for example, as analytical tools, as probes in biological assays, or as therapeutic agents in accordance with the present invention. The term “stereoisomer” refers to a molecule that is an enantiomer, diastereomer or geometric isomer of a molecule. Stereoisomers, unlike structural isomers, do not differ with respect to the number and types of atoms in the molecule's structure but with respect to the spatial arrangement of the molecule's atoms. Examples of stereoisomers include the (+) and (−) forms of optically active molecules.

As used herein, a “host cell” means a cell which contains an introduced nucleic acid construct and supports the replication and/or expression of the construct.

Having now described the inventive technology, the same will be illustrated with reference to certain examples, which are included herein for illustration purposes only, and which are not intended to be limiting of the invention.

### EXAMPLES

### Example 1: Biosynthesis of Squalene in Germinating Soybeans

The present inventors demonstrated the induction of biosynthesis of squalene in germinating soybeans as demonstrated in FIGS. 1 and 2 by HPLC chemical analysis. Dry soybeans were germinated with water and transfected with the ethanol inducible squalene epoxidase RNAi construct (SEQ ID NO. 1) by agrobacterium (K599). Ethanol was used to induce expression of squalene epoxidase RNAi by the ALCR/alcA ethanol switch system as described above. As shown in FIGS. 1 and 2, the induction of squalene epoxidase RNAi caused a significant increase of squalene produced in the germinating soybean root. Addition of the plant signaling hormone Methyl Jasmonate (MeJA) in conjunction with ethanol induced squalene epoxidase RNAi caused an additional increase in squalene production. Squalene was extracted from soybean tissue via 3:1 Isopropanol: Soybean (V/W) incubated overnight at 37C. Extract diluted 5× with distilled H2O, 75 uL injected into Agilent 1100 HPLC. HPLC Conditions-Poroshell 120 EC-C18 3 mm×75 mm 2.7 um; 45 minute linear 10-90% Buffer B gradient; Buffer A: 100% ddH2O; Buffer B: 100% Isopropanol.

### REFERENCES

1. Kajikawa, M., Kinohira, S., Ando, A., Shimoyama, M., Kato, M., & Fukuzawa, H. (2015). Accumulation of squalene in a microalga Chlamydomonas reinhardtii by genetic modification of squalene synthase and squalene epoxidase genes. PLoS One, 10(3), e0120446.
2. Nguyen, H. T., Neelakadan, A. K., Quach, T. N., Valliyodan, B., Kumar, R., Zhang, Z., & Nguyen, H. T. (2013). Molecular characterization of Glycine max squalene synthase genes in seed phytosterol biosynthesis. Plant Physiol Biochem, 73, 23-32.
3. Seo, J. W., Jeong, J. H., Shin, C. G., Lo, S. C., Han, S. S., Yu, K. W., Harada, E., Han, J. Y., & Choi, Y. E. (2005). Overexpression of squalene synthase in Eleutherococcus senticosus increases phytosterol and triterpene accumulation. Phytochemistry, 66(8), 869-877.
4. Wentzinger, L. F., Bach, T. J., & Hartmann, M. A. (2002). Inhibition of squalene synthase and squalene epoxidase in tobacco cells triggers an up-regulation of 3-hydroxy-3-methylglutaryl coenzyme a reductase. Plant Physiol, 130(1), 334-346.
5. Wu, S., Jiang, Z., Kempinski, C., Eric Nybo, S., Husodo, S., Williams, R., & Chappell, J. (2012). Engineering triterpene metabolism in tobacco. Planta, 236(3), 867-877.

1-13. (canceled)

14. A method of upregulating squalene production in a plant cell comprising:
expressing in a plant cell a heterologous nucleotide sequence, operably linked to a promoter, encoding a RNAi molecule directed against one or more of the plant's endogenous squalene epoxidase (SQE) enzymes, wherein said RNAi molecule downregulates the downstream conversion of squalene into sterols.

15. The method of claim 14, wherein said plant cell comprises a Glycine Max plant cell.

16. The method of claim 15, wherein said Glycine Max plant cell comprises a Glycine Max plant seed.

17. The method of claim 14, wherein said heterologous nucleotide sequence comprises a hairpin RNAi molecule targeting the expression of squalene epoxidase paralogs in Glycine Max.

18. The method of claim 17, wherein said hairpin RNAi comprises a hairpin RNAi molecule encoded by the nucleotide sequence according to SEQ ID NO. 1.

19. The method of claim 14, wherein said promoter comprises an inducible promoter

20. The method of claim 19, wherein said inducible promoter comprises an ALCR/alcA ethanol switch.

21. The method of claim 14, wherein the RNAi molecule comprises a nucleotide sequence according to SEQ ID NO. 1.

22. The method of claim 14, wherein said squalene epoxidase paralogs are selected from: SEQ ID NO's 5-14, or a fragment or variant thereof.

23. The method of claim 14, wherein said squalene epoxidase paralogs are selected from: SEQ ID NO's 5-7, and 9-12, or a fragment or variant thereof.

24. The method of claim 14, wherein said plant cell comprises a soybean seed derived from a soybean plant transformed by agrobacterium mediated transformation, or a soybean seed transformed by agrobacterium mediated transformation

25. A squalene compound produced by the method of claim 14.

26-31. (canceled)
